(19) United States
(12) Patent Application Publication
(10) Pub. No.: US 2018/0002765 A1
(43) Pub. Date: Jan. 4, 2018

(54) Molecular Marker Linked to Fusarium Wilt Resistance Gene in Tomato, Method for Obtaining the Same and Use Thereof

(71) Applicant: Ningxia Taijin Seed Co. Ltd., Zhongwei City (CN)

(72) Inventors: Weihao Zhou, Zhongwei City (CN); Jinglong Zhang, Zhongwei City (CN); Zhuoqiang Pei, Zhongwei City (CN); Qihai Shen, Zhongwei City (CN); Songzhen Shen, Zhongwei City (CN); Tiantian Zhai, Zhongwei City (CN)

(21) Appl. No.: 15/372,206

(22) Filed: Dec. 7, 2016

(30) Foreign Application Priority Data
Jul. 4, 2016 (CN) 201610514852.1

(51) Int. Cl. C12Q 1/68; A01H 1/02

(52) U.S. Cl. CPC C12Q 1/6895; C12Q 1/6848; A01H 1/02; C12Q 2600/156; C12Q 2600/158; C12Q 2600/13

(57) ABSTRACT

The present disclosure provides a molecular marker linked to Fusarium wilt resistance gene in tomato, and a method for obtaining the molecular marker linked to Fusarium wilt resistance gene in tomato. The molecular marker according to the present disclosure has a high specificity, and can be used to identify the resistance to Fusarium oxysporum f. sp. Physiological race 3 quickly and improve the breeding efficiency of the seeds resistant to Fusarium oxysporum f. sp. Physiological race 3. The molecular marker linked to Fusarium wilt resistance gene in tomato according to the present disclosure is prepared simply and has a low production cost. The molecular marker according to the present disclosure can be used to identify the resistance to Fusarium oxysporum f. sp. Physiological race 3, screen the tomato single plant which is resistant to Fusarium oxysporum f. sp. Physiological race 3, and/or determine the purity of hybrid seeds derived from hybridization of the tomato which is resistant to Fusarium oxysporum f. sp. Physiological race 3 and the tomato which is susceptible to Fusarium oxysporum f. sp. Physiological race 3. The molecular marker according to the present disclosure can be also used to prepare a kit, which has the same uses as that of the molecular marker according to the present disclosure.

### FIELD OF TECHNOLOGY

The present disclosure relates to the technical field of agricultural biotechnology, and in particular, relates to a molecular marker linked to Fusarium wilt resistance gene in tomato, a method for obtaining the same and use thereof.

### BACKGROUND

Fusarium oxysporum f. sp. Lycopersici, caused by Fusarium oxysporum f. sp. Lycopersici Snyder et Hansen, also referred to as Fusarium wilt, is a kind of disease that can wither, or even kill a whole tomato plant. Fusarium oxysporum f. sp. lycopersici race 3 is a physiological race that is highly toxic to tomato, can cause pathological wilting to the leaves of tomato, and even can cause tomato crops. Therefore, breeding of the tomatoes which are resistant to Fusarium oxysporum f. sp. lycopersici race 3 is a most direct and effective way to control Fusarium wilt.

The antigen of Fusarium oxysporum f. sp. lycopersici race 3 was discovered in Lycopersicon pennellii LA716, a tomato wild strain. The resistance of Lycopersicon pennellii LA716 was conferred by a single gene dominant inheritance, and the resistance gene is named as 13. At present, the resistance of tomato is identified generally by using molecular markers linked to resistance genes, then the resistance gene I3 is screened indirectly. The practicability of a molecular marker depends on the linkage strength of the molecular marker to the target gene and the utility cost of the molecular marker. The higher the linkage strength of the molecular marker to the target gene is, the closer the genetic distance is, the more accurate screening of the resistance gene I3 to Fusarium oxysporum f. sp. lycopersici race 3 is. However, the shortened genetic distance may result in a reduced number of available molecule markers, while most of the remaining available molecular markers are costly, such as SNP (Single Nucleotide Polymorphisms), CAPs (Cleaved Amplified Polymorphism Sequences), and the like, which will reduce the practicability of molecular markers. Therefore, during screening a molecular marker, the molecular marker, the genetic distance of which and the target gene is generally 5 cM (centi-morgan) or less, is generally selected. The marker SCAR (Sequence-characterized Amplified Region) is a specific primer marker of PCR (Polymerase Chain Reaction), which is convenient, efficient and reliable to detect a large number of individuals, and the detection result has a good stability and a high reproducibility. Therefore, the marker SCAR is a most preferable marker for the application of molecular markers in breeding practice.

At present, the SCAR marker commonly used for screening the resistance gene I3 is P7-43DF3/R1. The genetic distance between the marker P7-43DF3/R1 and I3 is less than 1 cM, so P7-43DF3/R1 can be used for the molecular mark assisted breeding of I3. However, as shown in the related literatures and the actual experimental results, the marker P7-43DF3/R1 can be only used to identify homozygous genetype I3/I3, cannot be used to distinguish heterozygote I3/i3 from homozygote i3/i3; and P7-43DF3/R1 has a poor specificity, which affects the selection of tomato resistant to Fusarium oxysporum f. sp. lycopersici race 3 and reduces the utilization efficiency of the marker SCAR.

### SUMMARY

An object of the present disclosure is to provide a molecular marker linked to Fusarium wilt resistance gene in tomato, a method for obtaining the same and use thereof, so as to solve the problem of poor specificity existed in the molecular marker P7-43DF3/R1 selecting the resistance gene I3.

The present disclosure provides a molecular marker linked to Fusarium wilt resistance gene in tomato, wherein the specific primer pair of the molecular marker is shown as follows:

forward primer:
(SEQ ID NO: 5)
5′-TTGTCAACCTTACCTTGCGTAC-3′;
and
reverse primer:
(SEQ ID NO: 6)
5′-AGGAACTTTATCACCATTGACA-3′.

The present disclosure provides a method for obtaining the molecular marker linked to Fusarium wilt resistance gene in tomato, which comprises the following steps:

obtaining primer sequences of a marker P7-43DF3/R1;

obtaining amplification sequences of the marker P7-43DF3/R1 in the processed tomato M82 and Lycopersicon pennellii by using BLAST, respectively;

comparing the homologous sequence of the amplification sequences in the processed tomato M82 and Lycopersicon pennellii, and designing primers at two ends of a polymorphic sequence; and

screening effective primers so as to obtain the molecular marker linked to Fusarium wilt resistance gene in tomato.

Preferably, the primer sequences of the marker P7-43DF3/R1 are shown as follows:

forward primer:
(SEQ ID NO: 3)
5′-CACGGGATATGTTATTGATAAGCATGT-3′;
and
reverse primer:
(SEQ ID NO: 4)
5′-GTCTTTACCACAGGAACTTTATCACC-3′.

Preferably, the amplification sequences of the marker P7-43DF3/R1 in the processed tomato M82 and Lycopersicon pennellii are SEQ ID NO:1 and SEQ ID NO:2 in the sequence listing, respectively.

The present disclosure provides use of the molecular marker linked to Fusarium wilt resistance gene in tomato, the molecular marker is used to identify the resistance to Fusarium oxysporum f. sp. Physiological race 3, screen the tomato single plant which is resistant to Fusarium oxysporum f. sp. Physiological race 3, and/or determine the purity of hybrid seeds hybridization of the tomato which is resistant to Fusarium oxysporum f. sp. Physiological race 3 and the tomato which is susceptible to Fusarium oxysporum f. sp. Physiological race 3.

The present disclosure provides a kitwhich comprises the molecular marker linked to Fusarium wilt resistance gene in tomato. The kit is used to identify the resistance to Fusarium oxysporum f. sp. Physiological race 3, screen the tomato single plant which is resistant to Fusarium oxysporum f. sp. Physiological race 3, and/or determine the purity of hybrid seeds derived from hybridization of the tomato which is resistant to Fusarium oxysporum f. sp. Physiological race 3 and the tomato which is susceptible to Fusarium oxysporum f. sp. Physiological race 3.

The present disclosure provides a method for identifying the resistance of tomato to Fusarium oxysporum f. sp. Physiological race 3, which comprises the following steps:

extracting DNA from a tomato to be tested;

amplifying the DNA of the tomato to be tested by PCR using the molecular marker linked to Fusarium wilt resistance gene in tomato as a primer, so as to obtain amplified products; and

subjecting the PCR amplified products to electrophoresis test and observing the band size of the PCR amplified products, wherein

if the band size of the PCR amplified products is 897 bp, the tomato to be tested has the resistance to Fusarium oxysporum f. sp. Physiological race 3; and

if the band size of the PCR amplified products is 687 bp, the tomato to be tested does not have the resistance to Fusarium oxysporum f. sp. Physiological race 3.

The present disclosure provides a method for screening the tomato single plant which is resistant to Fusarium oxysporum f. sp. Physiological race 3, which comprises the following steps:

subjecting susceptible tomatoes to crossbreeding with resistant tomatoes to obtain F1 generation, then subjecting the F1 generation to selfing to obtain the tomato single plants of F2 generation;

extracting DNA from the tomato single plants of F2 generation;

amplifying the DNA from the tomato single plants of F2 generation by PCR using the molecular marker linked to Fusarium wilt resistance gene in tomato as a primer, so as to obtain amplified products; and

subjecting the PCR amplified products to electrophoresis test and observing the band size of the PCR amplified product, wherein

if the band size of the PCR amplified products is 897 bp, the tomato single plants of F2 generation are homozygous single plants which are resistant to Fusarium oxysporum f. sp. Physiological race 3;

if the band size of the PCR amplified products is 897 bp+687 bp, the tomato single plants of F2 generation are heterozygous single plants which are resistant to Fusarium oxysporum f. sp. Physiological race 3; and

if the band size of the PCR amplified products is 687 bp, the tomato single plants of F2 generation are homozygous single plants which are susceptible to Fusarium oxysporum f. sp. Physiological race 3.

The present disclosure provides a method for determining the purity of hybrid seeds derived from hybridization of the tomato which is resistant to Fusarium oxysporum f. sp. Physiological race 3 and the tomato which is susceptible to Fusarium oxysporum f. sp. Physiological race 3, which comprises the following steps:

subjecting the female parent of susceptible tomatoes to crossbreeding with the male parent of resistant tomatoes so as to obtain tomato seeds of F1 generation;

extracting DNA of the tomato seeds of F1 generation;

amplifying the DNA from the tomato seeds of F1 generation by using PCR serving the molecular marker linked to Fusarium wilt resistance gene in tomato as a primer, so as to obtain amplified products;

subjecting the PCR amplified products to electrophoresis test and observing the band size of the PCR amplified products, wherein

if the band size of the PCR amplified products is double, the tomato seeds of F1 generation are hybrid seeds, and

if the band size of the PCR amplified products is single, the tomato seeds of F1 generation are selfed seeds; and

counting the numbers of hybrid seeds and selfed seeds respectively, calculating the ratio of the hybrid seed to the selfed seed to obtain the purity of hybrid seeds derived from hybridization of the tomato which is resistant to Fusarium oxysporum f. sp. Physiological race 3 and the tomato which is susceptible to Fusarium oxysporum f. sp. Physiological race 3.

Preferably, the conditions of PCR amplification are: 94° C., 5 min; 94° C., 30 s, 55° C., 30 s, 72° C., 45 s, for 33 cycles; 72° C., 10 min; held at 4° C.

The technical solutions provided by the embodiments of the present disclosure may bring out the following technical effects:

The present disclosure provides a molecular marker linked to Fusarium wilt resistance gene in tomato, the specific primer pair of the molecular marker is shown as follows: forward primer: 5′-TTGTCAACCTTACCTTGCGTAC-3′ (SEQ ID NO:5); and reverse primer: 5′-AGGAACTTTATCACCATTGACA-3′ (SEQ ID NO:6). The molecular marker linked to the Fusarium wilt resistance gene in tomato according to the present disclosure is a primer pair designed on the basis of the original molecular marker P7-43DF3/R1 selecting the resistance gene I3. By using the molecular marker linked to Fusarium wilt resistance gene in tomato according to the present disclosure as a primer, the DNA extracted from a tomato to be tested is amplified. After PCR amplification, the extracted DNA is subjected to electrophoresis test. The identification of the resistance to Fusarium oxysporum f. sp. Physiological race 3, the screening of the tomato single plant which is resistant to Fusarium oxysporum f. sp. Physiological race 3, and the determination of the purity of hybrid seeds derived from hybridization of the tomato which is resistant to Fusarium oxysporum f. sp. Physiological race 3 and the tomato which is susceptible to Fusarium oxysporum f. sp. Physiological race 3 are performed by counting the band numbers of the amplification products. The molecular marker according to the present disclosure has a high specificity, and can be used to identify the resistance to Fusarium oxysporum f. sp. Physiological race 3 quickly and improve the breeding efficiency of the seeds resistant to Fusarium oxysporum f. sp. Physiological race 3. The molecular marker linked to Fusarium wilt resistance gene in tomato according to the present disclosure is prepared simply and has a low production cost. The molecular marker according to the present disclosure can also be used to prepare a kit, and the kit can be used to identify the resistance to Fusarium oxysporum f. sp. Physiological race 3, screen the tomato single plant which is resistant to Fusarium oxysporum f. sp. Physiological race 3, and/or determine the purity of hybrid seeds derived from hybridization of the tomato which is resistant to Fusarium oxysporum f. sp. Physiological race 3 and the tomato which is susceptible to Fusarium oxysporum f. sp. Physiological race 3.

It should be understood that both the foregoing general description and the details later in the description are exemplary and explanatory, and cannot be construed to limit the present disclosure.

### BRIEF DESCRIPTION OF THE DRAWINGS

Figures, which herein are incorporated in and constitute a part of this specification, illustrate embodiments according to the present disclosure, and serve to explain the principles of the present disclosure together with the specification.

In order to more clearly illustrate the technical solutions according to the embodiments of the present disclosure or in prior arts, the drawings according to the embodiments of the present disclosure or prior arts will be described hereafter. It will be obvious for those skilled in the art that other drawings can also be obtained based on these drawings, without paying any creative effort.

FIG. 1 shows a flow chart illustrating a method for obtaining a molecular marker linked to Fusarium wilt resistance gene in tomato according to an embodiment of the present disclosure;

FIG. 2 shows a comparison chart illustrating the amplification sequences of the marker P7-43DF3/R1 in the processed tomato M82 and Lycopersicon pennellii according to an embodiment of the present disclosure;

FIG. 3 shows an electrophoretogram illustrating the tomato parent material amplified by using the molecular marker linked to tomato Fusarium wilt resistant gene according to the present disclosure as a primer;

FIG. 4 shows an electrophoretogram illustrating the single plants of F2 generation derived from the tomato parent resistant/susceptible to Fusarium oxysporum f. sp. Physiological race 3 amplified by using the molecular marker linked to tomato Fusarium wilt resistant gene according to the present disclosure as a primer; and

FIG. 5 shows an electrophoretogram illustrating the single plants of F1 generation derived from the tomato female parent resistant to Fusarium oxysporum f. sp. Physiological race 3 and the tomoato male parent susceptible to Fusarium oxysporum f. sp. Physiological race 3 amplified by serving the molecular marker linked to tomato Fusarium wilt resistant gene according to the present disclosure as a primer.

### DESCRIPTION OF THE EMBODIMENTS

The present disclosure hereafter will be further described with reference to the specific embodiments and the accompanying drawings. The following examples are provided to better understand the present disclosure, but cannot be construed to limit the present disclosure. Unless otherwise specified, the experimental methods described in examples below are conventional methods in the art, and all the drug materials used in the examples below are commercially available. The quantitative experiments in the examples below are carried out in triplicate, and the results are average values.

### Example 1: Obtaining a Molecular Marker Linked to Fusarium Wilt Resistance Gene in Tomato

The molecular marker linked to the Fusarium wilt resistance gene in tomato according to the present disclosure is a primer pair designed on the basis of the original SCAR marker P7-43DF3/R1 selecting the resistance gene I3, which has polymorphism and a high specificity. The molecular marker according to the present disclosure is named as TJ-1.

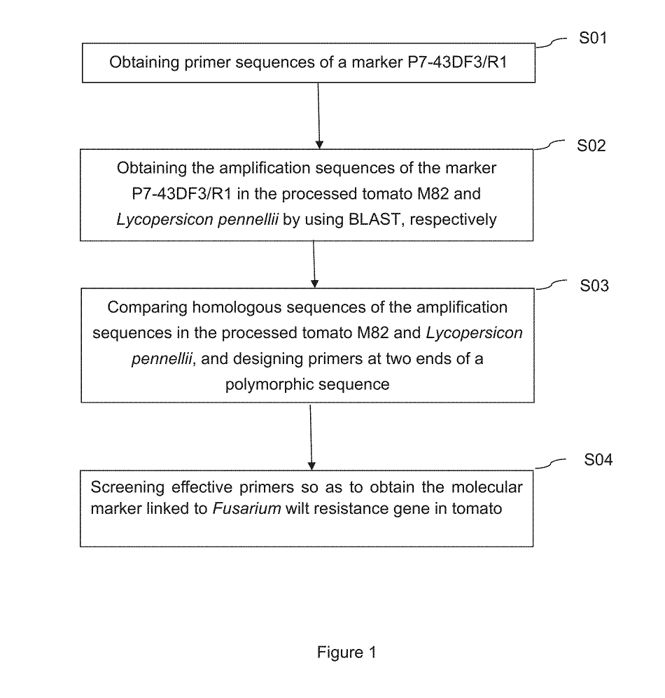

Refer to FIG. 1. FIG. 1 shows a flow chart illustrating a method for obtaining a molecular marker linked to Fusarium wilt resistance gene in tomato according to an embodiment of the present disclosure.

The method for obtaining the molecular marker linked to Fusarium wilt resistance gene in tomato according to the present disclosure comprises the following steps:

S01: The primer sequences of the marker P7-43DF3/R1 were obtained. The marker P7-43DF3/R1 is a common marker, so the primer sequences thereof are well known in the art, that is,

forward primer:
(SEQ ID NO: 3)
5′-CACGGGATATGTTATTGATAAGCATGT-3′;
and
reverse primer:
(SEQ ID NO: 4)
5′-GTCTTTACCACAGGAACTTTATCACC-3′.

S02: The amplification sequences of the marker P7-43DF3/R1 in the processed tomato M82 and Lycopersicon pennellii were obtained by using BLAST (Basic Local Alignment Search Tool) on the website NCBI (National Center for Biotechnology Information), respectively. In particular, the amplification sequences of the marker P7-43DF3/R1 in the processed tomato M82 and Lycopersicon pennellii are SEQ ID NO:1 and SEQ ID NO:2 in the sequence listing, respectively.

S03: The sequence cTOF-21-J12 in the processed tomato M82 and the homologous sequence Solanum pennellii chromosome ch07 in the amplification sequence of Lycopersicon pennellii were compared with sequence analysis software DNAMAN6.0, and primers were designed at two ends of a polymorphic sequence.

S04: The designed primers were screened to select the effective primers capable of performing PCR amplification so as to obtain the molecular marker TJ-1 linked to Fusarium wilt resistance gene in tomato. The primer sequences of the marker TJ-1 were shown as follows:

forward primer:
(SEQ ID NO: 5)
5′-TTGTCAACCTTACCTTGCGTAC-3′;
and
reverse primer:
(SEQ ID NO: 6)
5′-AGGAACTTTATCACCATTGACA-3′.

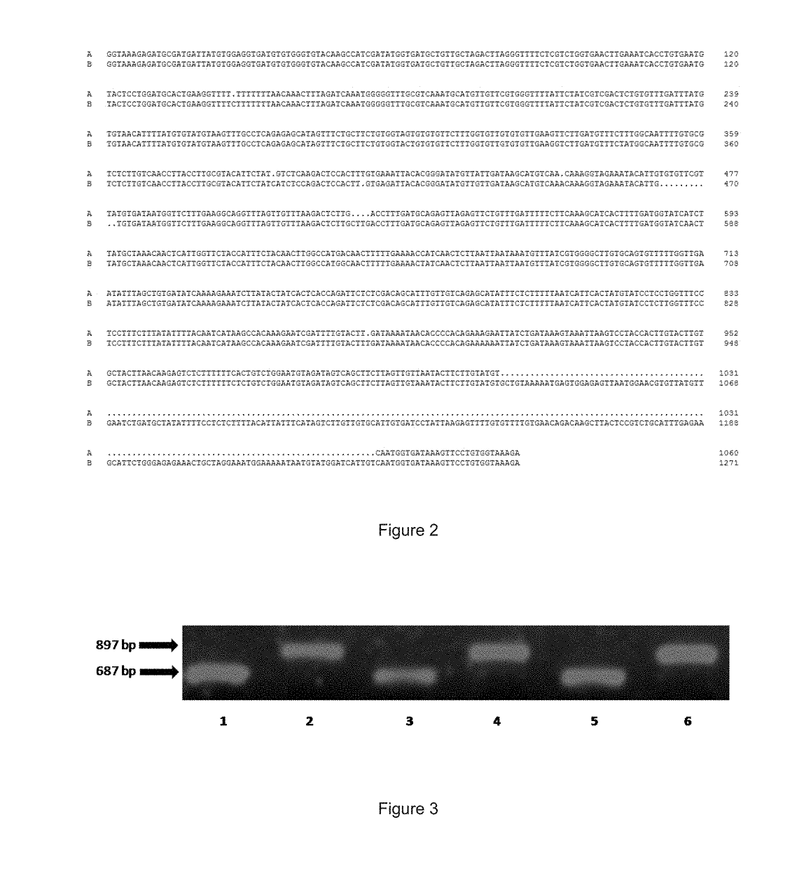

Refer to FIG. 2. FIG. 2 shows a comparison chart illustrating the amplification sequences of the marker P7-43DF3/R1 in the processed tomato M82 and Lycopersicon pennellii according to an embodiment of the present disclosure. A represents the amplification sequences of the marker P7-43DF3/R1 in the processed tomato M82, and B represents the amplification sequences of the marker P7-43DF3/R1 in Lycopersicon pennellii.

### Example 2

In order to test that the molecular marker TJ-1 according to the example of the present disclosure has a high specificity, i.e., the molecular marker TJ-1 according to the examples of the present disclosure can be used to identify the resistance to Fusarium oxysporum f. sp. Physiological race 3, the example of the present disclosure also provided three germplasm resources of tomato plants susceptible to Fusarium oxysporum f. sp. Physiological race 3 and three germplasm resources of tomato plants resistant to Fusarium oxysporum f. sp. Physiological race 3, wherein, Nos. 1, 3 and 5 represent tomato plants susceptible to Fusarium oxysporum f. sp. Physiological race 3, and Nos. 2, 4 and 6 represent tomato plants resistant to Fusarium oxysporum f. sp. Physiological race 3.

The method for identifying the resistance of tomato to Fusarium oxysporum f. sp. Physiological race 3 comprises the following steps.

The DNAs from the six tomato plants above were extracted, respectively.

The above six DNAs were amplified by using PCR serving the molecular marker TJ-1 as a primer, so as to obtain six amplified products.

The PCR amplified products were placed on 1% agarose gel, and subjected to electrophoresis test at a constant voltage 120V for 20 min. After stained with EB (Ethidium bromide), the PCR amplified products were placed in a gel imaging system to observe the sizes of the amplified bands in electrophoretogram.

If the band size of the PCR amplified products is 897 bp (base pair), the tomato to be tested has the resistance to Fusarium oxysporum f. sp. Physiological race 3.

If the band size of the PCR amplified products is 687 bp, the tomato to be tested does not have the resistance to Fusarium oxysporum f. sp. Physiological race 3.

Further, the method for extracting DNA from a tomato plant comprised the following steps. 10 mg of tomato young leaves was cut and placed into a 1.5 ml centrifuge tube, and 1000 ml of 0.25 mol/L NaOH solution was added thereto. Then the centrifuge tube was placed into boiling water for 30 s, and 1000 ml of 0.25 mol/L HCl solution was added thereto to neutralize NaOH. After completion of neutralization, 100 μL of 0.5 mol/L Tris-HCl solution was added into the centrifuge tube and the resultant solution was placed into boiling water for 2 min. Then the extract in the centrifuge tubes was taken out and the DNA of the tomato plant was contained in the extract. Obviously, the DNAs of the tomato plants can also be extracted by using well-known methods in the art, and the embodiments of the present disclosure do not limit the methods for extracting DNA.

When the molecular marker TJ-1 is used as a primer to perform PCR amplification, the system of the PCR amplification comprises 2×taq mix, 10 μL; forward primer of TJ-1, 0.4 μL; reverse primer of TJ-1, 0.4 μL; DNA extract, 2 μL; water, 7.2 μL. The conditions of PCR amplification are shown as follows: 94° C., 5 min; 94° C., 30 s, 55° C., 30 s, 72° C., 45 s, for 33 cycles; 72° C., 10 min; held at 4° C.

Refer to FIG. 3 for concrete results. FIG. 3 shows an electrophoretogram illustrating the parent material of tomato amplified by using the molecular marker linked to tomato Fusarium wilt resistant gene according to the present disclosure as a primer.

As can be seen from FIG. 3, after DNAs from tomatoes were amplified by using PCR serving the molecular marker TJ-1 linked to Fusarium wilt resistant gene as a primer, the band sizes of amplified tomato plants Nos. 1, 3 and 5 were 687 bp, while the band sizes of amplified tomato plants Nos. 2, 4 and 6 were 897 bp, which were consistent with the expected band sizes. Accordingly, the molecular marker TJ-1 linked to Fusarium wilt resistance gene in tomato according to the present disclosure is conformity with the results of the field identification, showing that the molecular marker TJ-1 according to the present disclosure has a good specificity and can be suitably used to identify the resistance to Fusarium oxysporum f. sp. Physiological race 3.

### Example 3

The molecular marker linked to Fusarium wilt resistance gene in tomato according to the present disclosure can not only be used to identify the resistance to Fusarium oxysporum f. sp. Physiological race 3, but also be used to screen the tomato single plant which is resistant to Fusarium oxysporum f. sp. Physiological race 3.

This example provided a method for screening the tomato single plant which is resistant to Fusarium oxysporum f. sp. Physiological race 3, comprising the following steps.

A susceptible tomato A was subjected to crossbreeding with a resistant tomato B to obtain F1 generation, then the F1 generation was subjected to self ing to obtain 24 tomato single plants of F2 generation.

DNAs were extracted from the tomato single plants of F2 generation.

The DNAs from the tomato single plants of F2 generation were amplified by using PCR serving the molecular marker TJ-1 as a primer, so as to obtain amplified products.

The PCR amplified products were placed on 1% agarose gel, and subjected to electrophoresis test at a constant voltage 120V for 20 min. After stained with EB, the PCR amplified products were placed in a gel imaging system to observe the size of the amplified bands in electrophoretogram.

If the band size of the PCR amplified products is 897 bp, the tomato single plants of F2 generation are homozygous single plants which are resistant to Fusarium oxysporum f. sp. Physiological race 3.

If the band size of the PCR amplified products is 897 bp+687 bp, the tomato single plants of F2 generation are heterozygous single plants which are resistant to Fusarium oxysporum f. sp. Physiological race 3.

If the band size of the PCR amplified products is 687 bp, the tomato single plants of F2 generation are homozygous single plants which are susceptible to Fusarium oxysporum f. sp. Physiological race 3.

The method for extracting DNAs of tomato plants, the system of PCR amplification and the conditions of PCR amplification in this example are the same as that in Example 2.

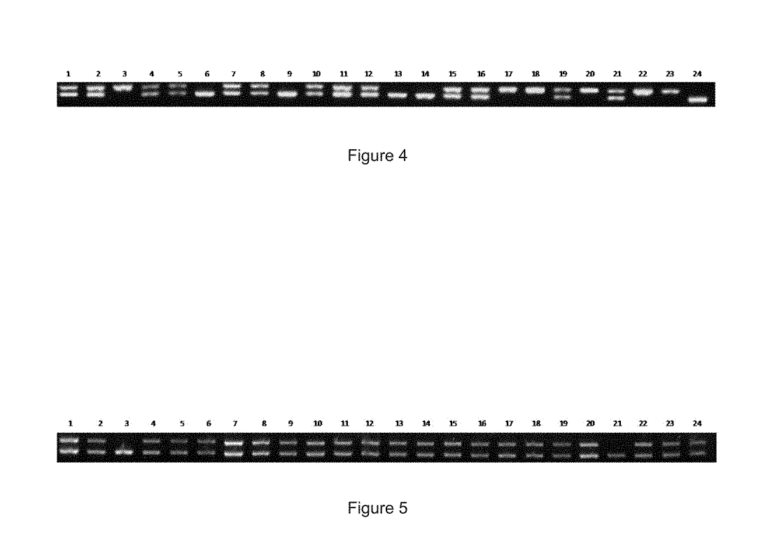

Refer to FIG. 4. FIG. 4 shows an electrophoretogram illustrating the single plants of generation F2 derived from the tomato parent resistant/susceptible to Fusarium oxysporum f. sp. Physiological race 3 amplified by using the molecular marker linked to tomato Fusarium wilt resistant gene according to the present disclosure as a primer. As can be seen from FIG. 4, after PCR amplification, the band size of tomato single plants Nos.: 3, 17, 18, 20, 22 and 23 was only 687 bp, the band size of tomato single plants Nos.: 6, 9, 13, 14, and 24 was 897 bp, and the band sizes of tomato single plants Nos.: 1, 2, 4, 5, 7, 8, 10, 11, 12, 13, 16, 19, and 21 were 897 bp and 687 bp. The results showed that the others were susceptible plants except that the tomato single plants Nos.: 3, 17, 18, 20, 22 and 23 were all resistant plants. 24 single plants of F2 generation were subjected to selfing, respectively, to obtain plants of generation F3, then the plants of F3 generation were subjected to inoculation test. The test results showed that all plants of F3 generation obtained by the selfing of the tomato single plants Nos.: 3, 17, 18, 20, 22 and 23 were infected with tomato Fusarium wilt, all the plants of F3 generation obtained by the selfing of the tomato single plants Nos.: 6, 9, 13, 14, and 24 were resistant to tomato Fusarium wilt, and the plants of F3 generation obtained by the selfing of the tomato single plants Nos.: 1, 2, 4, 5, 7, 8, 10, 11, 12, 13, 16, 19, and 21 showed resistant segregation, that is, the ratio of resistant tomatoes and susceptible tomatoes was 3:1. It also can be seen from FIG. 4 that the tomato single plants of F2 generation Nos.: 3, 17, 18, 20, 22 and 23 were homozygous susceptible single plants, the tomato single plants of F2 generation Nos.: 6, 9, 13, 14, and 24 were homozygous resistant single plants, and the tomato single plants of F2 generation Nos.: 1, 2, 4, 5, 7, 8, 10, 11, 12, 13, 16, 19, and 21 were heterozygous resistant single plants. In conclusion, the molecular marker TJ-1 according to this example of the present disclosure is capable of screening tomato single plant which is resistant to Fusarium oxysporum f. sp. Physiological race 3.

### Example 4

The molecular marker linked to Fusarium wilt resistance gene in tomato according to the present disclosure can also be used to determine the purity of hybrid seeds derived from hybridization of the tomato which is resistant to Fusarium oxysporum f. sp. Physiological race 3 and the tomato which is susceptible to Fusarium oxysporum f. sp. Physiological race 3.

This example provided a method for determining the purity of hybrid seeds derived from hybridization of the tomato which is resistant to Fusarium oxysporum f. sp. Physiological race 3 and the tomato which is susceptible to Fusarium oxysporum f. sp. Physiological race 3, comprising the following steps.

The female parent C of the susceptible tomato was subjected to crossbreeding with the male parent D of the resistant tomato so as to obtain 24 tomato seeds of F1 generation.

DNAs were extracted from the tomato seeds of F1 generation.

The DNAs from the tomato seeds of F1 generation were amplified by PCR using the molecular marker TJ-1 as a primer, so that amplified products were obtained.

The PCR amplified products were placed on 1% agarose gel, and subjected to electrophoresis test at a constant voltage 120V for 20 min. After stained with EB, the PCR amplified products were placed in a gel imaging system to observe the sizes of the amplified bands in electrophoretogram.

If the band of the PCR amplified products is double, the tomato seeds of F1 generation are hybrid seeds.

If the band of the PCR amplified products is single, the tomato seeds of F1 generation are selfed seeds.

The numbers of hybrid seeds and selfed seeds were counted respectively, and the ratio of the hybrid seed to the selfed seed was calculated to obtain the purity of hybrid seeds derived from hybridization of the tomato which is resistant to Fusarium oxysporum f. sp. Physiological race 3 and the tomato which is susceptible to Fusarium oxysporum f. sp. Physiological race 3.

SDS (sodium dodecyl sulfate) method was used to extract DNAs from the tomato seeds of F1 generation.

Refer to FIG. 5. FIG. 5 shows an electrophoretogram illustrating the single plants of generation F1 derived from the tomato female parent resistant to Fusarium oxysporumf. sp. Physiological race 3 and the tomoato male parent susceptible to Fusarium oxysporumf. sp. Physiological race 3 amplified by using the molecular marker linked to tomato Fusarium wilt resistant gene according to the present disclosure as a primer. As can be seen from FIG. 5, after PCR amplification, the band size of the tomato seeds Nos.: 3 and 21 was 687 bp, the band sizes of the other tomato seeds were 897 bp and 687 bp. Accordingly, the tomato seeds Nos.: 3 and 21 were homozygous susceptible seeds, and the remaining tomato seeds were heterozygous resistant seeds. The ratio of the resistant seeds in the seeds obtained from hybridization of the susceptible tomato female parent C and the resistant tomato female parent D was 22/24, that is, the purity of the seeds was 22/24. In conclusion, the molecular marker TJ-1 according to this example of the present disclosure is capable of rapidly determining the purity of hybrid seeds derived from hybridization of the tomato which is resistant to Fusarium oxysporum f. sp. Physiological race 3 and the tomato which is susceptible to Fusarium oxysporum f. sp. Physiological race 3.

As can be seen from Examples 2-4, the molecular marker TJ-1 linked to the Fusarium wilt resistance gene in tomato according to the examples of the present disclosure can be used to identify the resistance to Fusarium oxysporum f. sp. Physiological race 3 rapidly, screen the tomato single plant which is resistant to Fusarium oxysporum f. sp. Physiological race 3, and/or determine the purity of hybrid seeds derived from hybridization of the tomato which is resistant to Fusarium oxysporum f. sp. Physiological race 3 and the tomato which is susceptible to Fusarium oxysporum f. sp. Physiological race 3, so as to significantly improve the breeding efficiency of the seeds resistant to Fusarium oxysporum f. sp. Physiological race 3. At the same time, the molecular marker TJ-1 linked to Fusarium wilt resistance gene in tomato according to the present disclosure is prepared simply, has a low production cost, is applicable to a wide range of popularization and application and has a great application prospect.

Other examples of the invention will be readily apparent to those skilled in the art upon consideration of the specification and practices of the invention disclosed herein. This application is intended to encompass any variation, use or adaptations of the present disclosure that follow the general principles of the present disclosure and comprise common knowledge or conventional techniques that are not disclosed in the present disclosure. The specification and examples are considered as exemplary only, with the true scope and spirit of the disclosure being indicated by the appended claims.

It should be understood that the present disclosure is not limited to the precise constructions described above and illustrated in the accompanying drawings, various modifications and changes may be made without departing from its scope. The scope of the present disclosure is limited only by the appended claims.

What is claimed is:

1. A molecular marker linked to Fusarium wilt resistance gene in tomato, characterized in that, the specific primer pair of the molecular marker is shown as follows:
forward primer:
(SEQ ID NO: 5)
5′-TTGTCAACCTTACCTTGCGTAC-3′;
and
reverse primer:
(SEQ ID NO: 6)
5′-AGGAACTTTATCACCATTGACA-3′.

2. A method for obtaining the molecular marker linked to Fusarium wilt resistance gene in tomato according to claim 1, characterized in that, the method comprises the following steps:
obtaining primer sequences of a marker P7-43DF3/R1;
obtaining amplification sequences of the marker P7-43DF3/R1 in the processed tomato M82 and Lycopersicon pennellii by using BLAST, respectively;
comparing the homologous sequences of the amplification sequences in the processed tomato M82 and Lycopersicon pennellii, and designing primers at two ends of a polymorphic sequence; and
screening effective primers so as to obtain the molecular marker linked to Fusarium wilt resistance gene in tomato.

3. The method for obtaining the molecular marker linked to Fusarium wilt resistance gene in tomato according to claim 2, characterized in that, the primer sequences of the marker P7-43DF3/R1 are shown as follows:
forward primer:
(SEQ ID NO: 3)
5′-CACGGGATATGTTATTGATAAGCATGT-3′;
and
reverse primer:
(SEQ ID NO: 4)
5′-GTCTTTACCACAGGAACTTTATCACC-3′.

4. The method for obtaining the molecular marker linked to Fusarium wilt resistance gene in tomato according to claim 2, characterized in that, the amplification sequences of the marker P7-43DF3/R1 in the processed tomato M82 and Lycopersicon pennellii are SEQ ID NO:1 and SEQ ID NO:2 in the sequence listing, respectively.

5. Use of the molecular marker linked to Fusarium wilt resistance gene in tomato according to claim 1, characterized in that, the molecular marker is used to identify the resistance to Fusarium oxysporum f. sp. Physiological race 3 in tomato, screen the tomato single plant which is resistant to Fusarium oxysporum f. sp. Physiological race 3, and/or determine the purity of hybrid seeds derived from hybridization of the tomato which is resistant to Fusarium oxysporum f. sp. Physiological race 3 and the tomato which is susceptible to Fusarium oxysporum f. sp. Physiological race 3.

6. A kit, characterized in that, the kit comprises the molecular marker linked to Fusarium wilt resistance gene in tomato according to claim 1, the kit is used to identify the resistance to Fusarium oxysporum f. sp. Physiological race 3 in tomato, screen the tomato single plant which is resistant to Fusarium oxysporum f. sp. Physiological race 3, and/or determine the purity of hybrid seeds derived from hybridization of the tomato which is resistant to Fusarium oxysporum f. sp. Physiological race 3 and the tomato which is susceptible to Fusarium oxysporum f. sp. Physiological race 3.

7. A method for identifying the resistance to Fusarium oxysporum f. sp. Physiological race 3 in tomato, characterized in that, the method comprises the following steps:
extracting DNA from a tomato to be tested;
amplifying the DNA of the tomato to be tested by PCR using the molecular marker linked to Fusarium wilt resistance gene in tomato as a primer, so as to obtain amplified products; and
subjecting the amplified products to electrophoresis test and observing the band sizes of the PCR amplified products, wherein
if the band size of the PCR amplified products is 897 bp, the tomato to be tested has the resistance to Fusarium oxysporum f. sp. Physiological race 3; and
if the band size of the PCR amplified products is 687 bp, the tomato to be tested does not have the resistance to Fusarium oxysporum f. sp. Physiological race 3.

8. A method for screening a tomato single plant which is resistant to Fusarium oxysporum f. sp. Physiological race 3, characterized in that, the method comprises the following steps:
subjecting susceptible tomatoes to crossbreeding with resistant tomatoes to obtain F1 generation, then subjecting the F1 generation to selfing to obtain the tomato single plants of F2 generation;
extracting DNA from the tomato single plants of F2 generation;
amplifying the DNA from the tomato single plants of F2 generation by using PCR serving the molecular marker linked to Fusarium wilt resistance gene in tomato as a primer, so as to obtain amplified products; and
subjecting the amplified products to electrophoresis test and observing the band sizes of the PCR amplified products, wherein
if the band size of the PCR amplified products is 897 bp, the tomato single plants of F2 generation are homozygous single plants which are resistant to Fusarium oxysporum f. sp. Physiological race 3;
if the band size of the PCR amplified products is 897 bp+687 bp, the tomato single plants of F2 generation are heterozygous single plants which are resistant to Fusarium oxysporum f. sp. Physiological race 3; and
if the band size of the PCR amplified products is 687 bp, the tomato single plants of F2 generation are homozygous single plants which are susceptible to Fusarium oxysporum f. sp. Physiological race 3.

9. A method for determining the purity of hybrid seeds derived from hybridization of the tomato which is resistant to Fusarium oxysporum f. sp. Physiological race 3 and the tomato which is susceptible to Fusarium oxysporum f. sp. Physiological race 3, characterized in that, the method comprises the following steps:
subjecting a female parent of susceptible tomatoes to crossbreeding with a male parent of resistant tomatoes so as to obtain tomato seeds of F1 generation;
extracting DNA from the tomato seeds of F1 generation;
amplifying the DNA from the tomato seeds of F1 generation by using PCR serving the molecular marker linked to Fusarium wilt resistance gene in tomato as a primer, so as to obtain amplified products;
subjecting the amplified products to electrophoresis test and observing the band sizes of the PCR amplified products, wherein
if the band size of the PCR amplified products is double, the tomato seeds of F1 generation are hybrid seeds, and
if the band size of the PCR amplified products is single, the tomato seeds of F1 generation are selfed seeds; and
counting the numbers of the hybrid seeds and the selfed seeds respectively, calculating the ratio of the hybrid seeds to the selfed seeds to obtain the purity of the hybrid seeds derived from hybridization of the tomato which is resistant to Fusarium oxysporum f. sp. Physiological race 3 and the tomato which is susceptible to Fusarium oxysporum f. sp. Physiological race 3.

10. The method according to claim 7, characterized in that, the conditions of PCR amplification are: 94° C., 5 min; 94° C., 30 s, 55° C., 30 s, 72° C., 45 s, for 33 cycles; 72° C., 10 min; and held at 4° C.

11. The method according to claim 8, characterized in that, the conditions of PCR amplification are: 94° C., 5 min; 94° C., 30 s, 55° C., 30 s, 72° C., 45 s, for 33 cycles; 72° C., 10 min; and held at 4° C.

12. The method according to claim 9, characterized in that, the conditions of PCR amplification are: 94° C., 5 min; 94° C., 30 s, 55° C., 30 s, 72° C., 45 s, for 33 cycles; 72° C., 10 min; and held at 4° C.


SEQUENCE LISTING (6 sequences)

SEQ ID NO: 1 - DNA, 1061 bp, Lycopersicon esculentum
ggtaaagagatgcgatgattatgtggaggtgatgtgtgggtgtacaagccatcgatatggtgatgctgttgctagacttagggttttctcgtctggtgaacttgaaatcacctgtgaatgtactcctggatgcactgaaggtttttttttttaacaaactttagatcaaatgggggtttgcgtcaaatgcatgttgttcgtgggttttattctatcgtcgactctgtgtttgatttatgtgtaacattttatgtgtatgtaagtttgcctcagagagcatagtttctgcttctgtggtagtgtgtgttctttggtgttgtgtgttgaagttcttgatgtttctttggcaattttgtgcgtctcttgtcaaccttaccttgcgtacattctatgtctcaagactccactttgtgaaattacacgggatatgttattgataagcatgtcaacaaaggtagaaatacattgtgtgttcgttatgtgataatggttctttgaaggcaggtttagttgtttaagactcttgacctttgatgcagagttagagttctgtttgatttttcttcaaagcatcacttttgatggtatcatcttatgctaaacaactcattggttctaccatttctacaacttggccatgacaactttttgaaaaccatcaactcttaattaataaatgtttatcgtggggcttgtgcagtgtttttggttgaatatttagctgtgatatcaaaagaaatcttatactatcactcaccagattctctcgacagcatttgttgtcagagcatatttctctttttaatcattcactatgtatcctcctggtttcctcctttctttatattttacaatcataagccacaaagaatcgattttgtacttgataaaataacaccccacagaaagaattatctgataaagtaaattaagtcctaccacttgtacttgtgctacttaacaagagtctcttttttcactgtctggaatgtagatagtcagcttcttagttgttaatacttcttgtatgtcaatggtgataaagttcctgtggtaaagac

SEQ ID NO: 2 - DNA, 1272 bp, Lycopersicon esculentum
ggtaaagagatgcgatgattatgtggaggtgatgtgtgggtgtacaagccatcgatatggtgatgctgttgctagacttagggttttctcgtctggtgaacttgaaatcacctgtgaatgtactcctggatgcactgaaggttttctttttttaacaaactttagatcaaatgggggtttgcgtcaaatgcatgttgttcgtgggttttattctatcgtcgactctgtgtttgatttatgtgtaacattttatgtgtatgtaagtttgcctcagagagcatagtttctgcttctgtggtactgtgtgttctttggtgttgtgtgttgaaggtcttgatgtttctatggcaattttgtgcgtctcttgtcaaccttaccttgcgtacattctatcatctccagactccacttgtgagattacacgggatatgttgttgataagcatgtcaaacaaaggtagaaatacattgtgtgataatggttctttgaaggcaggtttagttgtttaagactcttgcttgacctttgatgcagagttagagttctgtttgatttttcttcaaagcatcacttttgatggtatcaacttatgctaaacaactcattggttctaccatttctacaacttggccatggcaactttttgaaaactatcaactcttaattaattaatgtttatcgtggggcttgtgcagtgtttttggttgaatatttagctgtgatatcaaaagaaatcttatactatcactcaccagattctctcgacagcatttgttgtcagagcatatttctctttttaatcattcactatgtatcctcttggtttcctcctttctttatattttacaatcataagccacaaagaatcgattttgtactttgataaaataacaccccacagaaaaaattatctgataaagtaaattaagtcctaccacttgtacttgtgctacttaacaagagtctcttttttctctgtctggaatgtagatagtcagcttcttagttgtaaatacttcttgtatgtgctgtaaaaatgagtggagagttaatggaacgtgttatgttgaatctgatgctatattttcctctcttttacattatttcatagtcttgttgtgcattgtgatcctattaagagttttgtgttttgtgaacagacaagcttactccgtctgcatttgagaagcattctgggagagaaactgctaggaaatggaaaaataatgtatggatcattgtcaatggtgataaagttcctgtggtaaagac

SEQ ID NO: 3 - DNA, 27 bp, Artificial sequence
  Synthesized sequence
cacgggatatgttattgataagcatgt

SEQ ID NO: 4 - DNA, 26 bp, Artificial sequence
  Synthesized sequence
gtctttaccacaggaactttatcacc

SEQ ID NO: 5 - DNA, 22 bp, Artificial sequence
  Synthesized sequence
ttgtcaaccttaccttgcgtac

SEQ ID NO: 6 - DNA, 22 bp, Artificial sequence
  Synthesized sequence
aggaactttatcaccattgaca